SEQUENCE LISTING (6 sequences)

SEQ ID NO: 1 - DNA, 24 bp, Artificial Sequence
  forward primer pCVP18-F1
tagtcgacaattatggcttcatcg

SEQ ID NO: 2 - DNA, 20 bp, Artificial Sequence
  reverse primer pCVP18-R2
accggaattccatggcaatg

SEQ ID NO: 3 - DNA, 25 bp, Artificial Sequence
  primer pCVP18-F7
cattgccatggaattccggtttact

SEQ ID NO: 4 - DNA, 26 bp, Artificial Sequence
  reverse primer pCVP18-R1
gcggccgctcaggttgttggtcttgt

SEQ ID NO: 5 - DNA, 31 bp, Artificial Sequence
  primer GA1F4
taccgcggaattatgtctcttcagtatcatg

SEQ ID NO: 6 - DNA, 27 bp, Artificial Sequence
  primer GA1NotIR
gcggccgcctagactttttgaaacaag

(19) United States
(12) Patent Application Publication
(10) Pub. No.: US 2011/0177552 A1
(43) Pub. Date: Jul. 21, 2011

(54) SYSTEMS AND METHODS FOR PRODUCING PLASTID PROTEINS

(76) Inventors: Caroline Virginia McNeil, Houston, TX (US); Alyssa Baevich, Richmond, TX (US); Seiichi Paul Tillich Matsuda, Houston, TX (US)

(21) Appl. No.: 13/008,739

(22) Filed: Jan. 18, 2011

Related U.S. Application Data
(63) Continuation of application No. PCT/US09/51014, filed on Jul. 17, 2009.
(60) Provisional application No. 61/081,792, filed on Jul. 18, 2008.

(51) Int. Cl. C12P 21/06; C12N 9/88; C12P 5/00; C12N 9/02

(52) U.S. Cl. 435/68.1; 435/232; 435/166; 435/189

(57) ABSTRACT

Methods and systems that include a method comprising: providing a system comprising one or more plastid proteins comprising a signal sequence and one or more chloroplast processing enzymes; and allowing at least one of the one or more chloroplast processing enzymes to cleave at least a portion of a signal sequence from at least one of the one or more plastid proteins.

### CROSS-REFERENCE TO RELATED APPLICATIONS

This application is a continuation of International Application No. PCT/US09/051014, filed Jul. 17, 2009, which claims the benefit of U.S. Provisional Patent Application Ser. No. 61/081,792, filed Jul. 18, 2008, the entire disclosures of which are incorporated by reference.

### BACKGROUND

The present invention relates generally to protein production. In particular, the present invention relates to systems and methods for producing plastid proteins.

Biosynthesis of diterpenes in plants occurs in plastids, and therefore diterpene synthases usually contain N-terminal transit peptide signal sequences directing them to the plastids. (1-4) Once in the organelle, the transit sequences are cleaved to form the mature, functional diterpene synthases. The initial full-length protein containing the transit peptide sequence expressed in E. coli has poor enzymatic activity, whereas removal of the signal sequence has been demonstrated to increase enzymatic activity and aid in enzyme characterization. (5-7)

Pseudomature proteins are heterologously expressed proteins lacking the transit peptide. These enzymes are generated by making a subclone without the signal sequence, but with an artificial start site introduced at the beginning of the expected mature protein. Pseudomature proteins differ from conventional mature proteins because they retain this adventitious methionine, and pseudomature diterpene synthases often have better activity than their full-length counterparts when expressed in E. coli. (6,8,9) However, the exact location of the cleavage site may not be readily predictable, therefore extensive empirical experiments are often necessary to determine the best site for N-terminal truncation. (6,8,10,11) Moreover, one may not be certain that the introduced methionine in the pseudomature protein will not alter catalysis in some manner. However, plants encode proteins known as chloroplast processing enzymes (CPE), which cleave transit peptides once proteins enter the plastid. (12)

While much of the description and examples herein pertains to the use of the systems and methods of the present invention for diterpene production, such is not intended to limit the scope of the present invention. Rather, the systems and methods disclosed herein may be applicable to any suitable compound which may be produced by one or more plastid proteins.

### DRAWINGS

Some specific example embodiments of the disclosure may be understood by referring, in part, to the following description and the accompanying drawings.

FIG. 1 shows a reaction scheme for cyclization of GGPP to abietadiene by abietadiene synthase.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

The present invention relates generally to protein production. In particular, the present invention relates to systems and methods for producing plastid proteins.

The present disclosure provides, in certain embodiments, a method comprising providing a system comprising one or more plastid proteins and one or more chloroplast processing enzymes; and allowing at least one of the one or more chloroplast processing enzymes to cleave at least a portion of a signal sequence from at least one of the one or more plastid proteins.

The present disclosure provides, in certain embodiments, a system comprising one or more plastid proteins and one or more chloroplast processing enzymes.

In certain embodiments, the systems and methods of the present invention may confer benefits beyond traditional methods of plastid protein production. Such traditional methods include extraction from natural sources and chemical synthesis. In certain embodiments, the systems and methods of the present invention may increase the catalytic activity of the resulting plastid proteins. In certain embodiments, the systems and methods of the present invention may increase the yield of a product or products produced by the resulting plastid proteins. In certain embodiments, the cleavage of at least a portion of a signal sequence from at least one of the one or more plastid proteins may increase the solubility in water of the plastid protein, which may contribute in part to the increased catalytic activity of the plastid protein and/or the increase yield of a product or products produced by the resulting plastid proteins.

The plastid proteins useful in the systems and methods of the present invention may be any plastid protein which exhibits at least partially altered properties in an untruncated state. As used herein, the term “untruncated” and its derivatives mean that the plasmid protein contains one or more amino acids which must be removed to allow the protein to function as desired. Such untruncated plasmid proteins may include, but are not limited to, plasmid proteins which contain a signaling sequence. In certain embodiments, such a signaling sequence may inhibit the ability of the plastid protein to perform a desired function, such as, but not limited to, the catalysis of a chemical reaction. In certain embodiments, the plastid protein may be a diterpene synthase.

The chloroplast processing enzymes useful in the systems and methods of the present invention may be derived from any suitable source. Suitable sources include, but are not limited to, plants such as Arabidopsis thaliana and Abies grandis. In certain embodiments, the chloroplast processing enzyme may be derived from the same organism as the plastid proteins. In certain embodiments, using chloroplast processing enzymes and plastid proteins from the same organism may increase the catalytic activity of the resulting plastid proteins and/or the yield of a product or products produced by the resulting plastid proteins when compared to the catalytic activity of the resulting plastid proteins and/or the yield of a product or products produced by the resulting plastid proteins when the chloroplast processing enzyme and plastid proteins are derived from different organisms.

In certain embodiments, the systems and methods of the present invention may allow for the increased yield of a product derived from the plastid proteins useful in the systems and methods of the present invention. In certain embodiments, the plastid proteins may comprise one or more enzymes, and the systems and methods of the present invention may allow for the increased yield of the reaction products of the plastid protein with one or more substrates. For example, in certain embodiments where the plastid proteins comprise one or more diterpene synthases, the systems and methods of the present invention may increase the yield of diterpene hydrocarbons. In such embodiments, the systems of the present invention may comprise proteins which produce diterpene precursors. Such proteins include, but are not limited to, geranylgeranyl diphosphate synthase and hydroxymethylglutaryl CoA reductase. Desirable diterpenes which may be produced by such embodiments of the systems and methods of the present invention include, but are not limited to, paclitaxel.

In certain embodiments, the systems and methods of the present invention may be contained within a microorganism. Suitable microorganisms include, but are not limited to, E. coli. In certain embodiments, the plastid proteins and/or chloroplast processing enzymes useful in the systems and methods of the present invention may be introduced into the microorganism. In certain embodiments, the plastid proteins and/or chloroplast processing enzymes useful in the systems and methods of the present invention may be produced by the microorganism, for example, by introducing the genes encoding the plastid proteins and/or chloroplast processing enzymes into the genome of the microorganism.

In certain embodiments, additional enzymes may be added to the systems of the present invention which metabolize the products produced by the plastid proteins (such as diterpenes) further. Such additional enzymes include, but are not limited to, P450-dependent oxidases. In certain embodiments, the inclusion of such additional enzymes may provide a larger library of molecules that could be produced by the systems and methods of the present invention.

In certain embodiments, one or more competing enzymes may be suppressed. The term “competing enzyme” is used herein to mean an enzyme which would hinder the production of the desired product. Such competing enzymes may hinder the production of the desired product by one or more of the traditional methods of enzymatic competition. In certain embodiments where the systems and methods of the present invention are used to produce diterpenes, squalene synthase may be suppressed. Such suppression may hinder the consumption of carbon by squalene synthase and allow for increase diterpene production.

As previously stated, while much of the description and examples herein pertains to the use of the systems and methods of the present invention for diterpene production, such is not intended to limit the scope of the present invention. Rather, the systems and methods disclosed herein may be applicable to any suitable compound which may be produced by one or more plastid proteins. For example, many monoterpenes also may be produced in the plastids and a large number of monoterpene synthases contain a signaling sequence. Co-expression of a chloroplast processing enzyme with a monoterpene synthase may increase monoterpene yields in vivo.

To facilitate a better understanding of the present invention, the following examples of specific embodiments are given. In no way should the following examples be read to limit or define the entire scope of the invention.

### EXAMPLES

### Experimental Procedures

RT-PCR to obtain a cDNA

mRNA from 7-day Arabidopsis seedlings (Columbia) was obtained, and reverse-transcription PCR was performed following the manufacturer's protocol to obtain a cDNA (Ambion, Austin, Tex.).

PCR amplification of CPE from cDNA

The full-length Arabidopsis CPE gene sequence (At5g42390) was obtained from NCBI and used to develop primers for PCR amplification from the cDNA library. Because of the length of the CPE gene (3.8 kB), it was necessary to amplify the gene in two parts: from Sal I to EcoR I, and from EcoR I to Not I. For the first fragment, the PCR mixture was as follows: 1 μL 7-day seedling cDNA, 1 μL of the forward primer pCVP18-F1 5′-TAGTCGACAATTATGGCTTCATCG-3′ (SEQ ID NO. 1) (Sal I restriction site underlined), 1 μL of the reverse primer pCVP18-R2 5′-ACCGGAATTCCATGGCAATG-3′ (SEQ ID NO. 2) (EcoR I restriction site underlined), 4 μL dNTPs, 5 μL High-Fidelity buffer, 0.2 μL Triple Master polymerase, and 38 μL mqH₂O. The back fragment contained the same mixture, except the primers used were pCVP18-F7 5′-CATTGCCATGGAATTCCGGTTTACT-3′ (SEQ ID NO. 3) (EcoR I restriction site underlined) and pCVP18-R1 5′-GCGGCCGCTCAGGTTGTTGGTCTTGT-3′(SEQ ID NO. 4) (Not I restriction site underlined).

### Construction of the Full-Length CPE-Containing Plasmid

The two CPE fragments, Sal I to EcoR I, and EcoR I to Not I, were ligated into TOPO-TA vector, and clones containing the inserts were sequenced. Large-scale cultures of clones with the correct sequences were grown and DNA isolated, yielding two plasmids pAMB3.0 and pAMB4.0, corresponding to the front and back pieces of the CPE, respectively.

The two plasmids pAMB3.0 and pAMB4.0 were digested with their corresponding restriction enzymes and ligated into pRS313Gal that had been digested with Sal I and Not I. The three-piece ligation was then transformed into E. coli and plated on LB+Amp. Clones were screened for the presence of the insert, and the correct clone was sequenced to ensure the full-length gene was obtained with the correct sequence. The final plasmid was named pAMB5.3.

### Subcloning GA1 and GA2 Into One Plasmid

A plasmid containing both Arabidopsis GA1 and GA2 was constructed. First, GA2 and a bi-directional galactose (BiGal) promoter were subcloned into pRS426 with BamH I and Not I to give the plasmid pCVP6.3. GA1 was PCR-amplified to insert new restriction sites using the primers GA1F4 5′-TACCGCGGAATTATGTCTCTTCAGTATCATG-3′ (SEQ ID NO. 5) and GA1NotIR5′-GCGGCCGCCTAGACTTTTTGAAACAAG_-3′ (SEQ ID NO. 6). The gel-purified PCR product was cloned into pCVP6.3 with Sac II and Not I, resulting in the final plasmid pCVP26.1.

### Construction of Yeast Strains

Two control strains were first constructed. EHY18 was transformed with either the pCVP26.1 plasmid or pEH9.0, the Abies grandis abietadiene synthase in pRS426Gal. These two yeast strains, EHY18[pCVP26.1] and EHY18[pEH9.0], were selected on synthetic complete media lacking uracil and transformed with the pAMB5.3 plasmid. The final transformants were screened by growing on synthetic complete media lacking both uracil and histidine.

### Identification and Quantitation of Products Generated in Yeast Strains

All strains were grown to saturation in inducing medium and harvested by centrifugation. The cell pellets were saponified and extracted with 3×15 mL hexanes, with 100 μL of the internal standard epicoprostanol (2.5 mg in 1 mL ethanol) added prior to saponification. The non-saponifiable lipids (NSL) were partitioned between one-half volume water and 3×15 mL hexanes, and solvent was evaporated in vacuo and residues transferred to GC vials with CH₂Cl₂. Samples were analyzed on GC-FID and GC-MS.

### Results

The Arabidopsis chloroplast processing enzyme (CPE, At5g42390) was PCR-amplified from a cDNA and co-expressed in EHY18 containing either the Abies grandis (grand fir) abietadiene synthase (AgAS) or the Arabidopsis ent-copalyl pyrophosphate synthase (AtGA1) and ent-kaurene synthase (A tGA2) genes (Table 1). (13-17) Control strains expressing only diterpene synthases were grown in parallel, and the diterpene products accumulating in the cell pellets and media were analyzed. Triplicate cultures were grown and harvested to provide more accurate data.

TABLE 1
Summary of yeast strains with and without CPE.

| Yeast strain | Characteristics |
|---|---|
| EHY18[pEH9.0] | AgAS::URA3 |
| EHY18[pEH9.0][pAMB5.3] | AgAS::URA3 AtCPE::HIS3 |
| EHY18[pCVP26.1] | AtGA1 + AtGA2::URA3 |
| EHY18[pCVP26.1][pAMB5.3] | AtGA1 + AtGA2::URA3 AtCPE::HIS3 |

All yeast strains contain MATa pGAL1-BTS1::hisG pGAL1-trHMG1::LEU2 ura3-52 trp1-Δ63 leu2-3,112 his3-Δ200 ade2 Gal⁺.

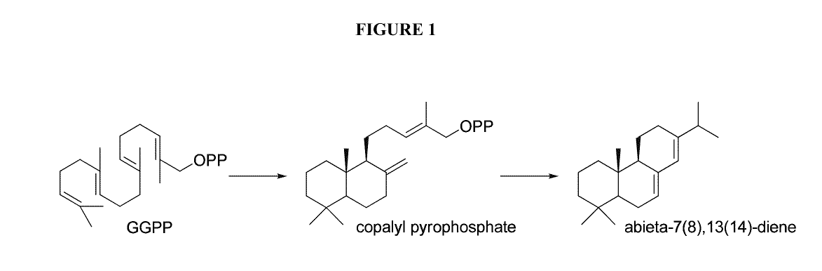

The cell pellets from EHY18[pEH9.0] and EHY18[pEH9.0][pAMB5.3] were saponified, and the non-saponifiable lipids (NSL) contained the majority of the diterpene products (Table 2). The NSL were thus used for quantitation, as insignificant levels of diterpene products were isolated from the media (<1% of the total). GC-MS analysis of the NSL showed that the cultures produced abietadiene as the major diterpene product. Abietadiene synthase was previously reported to catalyze cyclization of geranylgeranyl pyrophosphate (GGPP) to the intermediate (+)-copalyl pyrophosphate, which is then cyclized further to (−)-abieta-7(8),13(14)-diene (FIG. 1).

TABLE 2
GC-FID quantitation of EHY18[pEH9.0]
NSL with and without CPE co-expression.

| | | Geranyl- | |
|---|---|---|---|
| | Abietadiene | geraniol | Squalene |
| Strain | (mg/L) | (mg/L) | (mg/L) |
| EHY18[pEH9.0] | 5.91 ± 0.55 | N.D. | 14.18 ± 2.16 |
| EHY18[pEH9.0] | 9.11 ± 0.58 | N.D. | 8.29 ± 1.02 |
| [pAMB5.3] | | | |

Peak areas were compared to that of the internal standard epicoprostanol.

The amount of abietadiene isolated from the CPE-expressing strain increased 1.5 times in comparison to the strain without the CPE. Both strains accumulated a significant amount of abietadiene, with nearly 6 mg/L in the strain without the CPE and over 9 mg/L in the strain with the CPE. Both the control strain and the strain co-expressing the CPE produced abietadiene but did not accumulate substantial levels of the GGPP hydrolysis products geranylgeraniol or geranyllinalool.

Because the yeast strains have intact sterol biosynthetic pathways, squalene was also isolated but at much higher levels than the diterpene products. In the strain without the CPE, approximately 14 mg/L of squalene was accumulated, suggesting that the amount of carbon flux was too great for the enzymes in the sterol pathway to process it efficiently. The strain with the co-expressed CPE, however, demonstrated a large decrease in squalene accumulation, with only 8.3 mg/L recovered.

Usually, most FPP funnels through the sterol biosynthetic pathway instead of being used for GGPP production. Over-expression of GGPP synthase shunted more FPP to GGPP biosynthesis and therefore provided more substrate for diterpene synthesis, decreasing the amount of carbon routed towards sterol biosynthesis. (13)

More dramatic changes were observed in the strains co-expressing GA1 and GA2 with the CPE (Table 3). As seen in the strains containing abietadiene synthase, almost all of the ent-kaurene was isolated from the cell pellets, with less than 1% of the ent-kaurene found in the media. From these results, the amount of ent-kaurene accumulating in the CPE co-expression strain was 10 times higher than that observed in the strain without the CPE. The parent strain accumulated approximately 0.25 mg/L ent-kaurene, while the strain with the CPE accumulated approximately 2.5 mg/L.

TABLE 3
GC-FID quantitation of EHY18[pCVP26.1]
NSL with and without CPE co-expression.

| | | Geranyl- | |
|---|---|---|---|
| | ent-Kaurene | geraniol | Squalene |
| Strain | (mg/L) | (mg/L) | (mg/L) |
| EHY18[pCVP26.1] | 0.25 ± 0.08 | 3.54 ± 1.26 | 8.18 ± 1.85 |
| EHY18[pCVP26.1] | 2.47 ± 1.34 | 2.46 ± 0.63 | 6.00 ± 0.73 |
| [pAMB5.3] | | | |

Peak areas were compared to that of the internal standard epicoprostanol.

The CPE co-expression greatly improved GA1 and GA2 activity, as shown by the substantial difference in ent-kaurene accumulation. No significant amounts of the ent-kaurene intermediates ent-copalol or ent-manool/ent-13-epimanool were observed (<1% of ent-kaurene), suggesting that GA2 enzymatic activity greatly increased and more efficiently cyclized ent-copalyl pyrophosphate to ent-kaurene.

In addition to ent-kaurene, a considerable amount of geranylgeraniol was also isolated, with approximately 3.0 mg/L accumulating in both EHY18[pCVP26.1] and the CPE-expressing strain. The large amount of geranylgeraniol isolated suggests that the GA1 and GA2 enzymes were not as efficient as the abietadiene synthase. As observed in the abietadiene strains, the amount of squalene accumulation was substantial, with over 8 mg/L of squalene isolated from EHY18[pCVP26.1]. The amount of squalene decreased significantly to 6 mg/L when the CPE was expressed. ent-Kaurene production increased at the same rate that squalene accumulation decreased, again consistent with CPE increasing GA1 and GA2 activity.

Co-expressing Arabidopsis CPE improved abietadiene production somewhat less than it did in the ent-kaurene-producing strains, consistent with better cleavage when CPE and cyclase both come from Arabidopsis than when they are from different organisms. This difference may reflect the evolutionary distance between A. grandis and Arabidopsis. The CPE substrate recognition sites may not be similar enough to efficiently cleave the transit peptide from the other organism.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

### References:

The following references are all incorporated by reference to the extent they provide information available to one of ordinary skill in the art regarding the implementation of the technical teachings of the invention.
1. Dudley, M. W.; Dueber, M. T.; West, C. A. Plant Physiol. 1986, 81, 335-342.
2. Moore, T. C.; Coolbaugh, R. C. Phytochemistry 1976, 15, 1241-1247.
3. Railton, I. D.; Fellows, B.; West, C. A. Phytochemistry 1984, 23, 1261-1267.
4. Bohlmann, J.; Meyer-Gauen, G.; Croteau, R. Proc. Natl. Acad. Sci. U. S. A. 1998, 95, 4126-4133.
5. LaFever, R. E.; Stofer Vogel, B.; Croteau, R. Arch. Biochem. Biophys. 1994, 313, 139-149.
6. Williams, D. C.; Wildung, M. R.; Jin, A. Q.; Dalal, D.; Oliver, J. S.; Coates, R. M.; Croteau, R. Arch. Biochem. Biophys. 2000, 379, 137-146.
7. Huang, K. X.; Huang, Q. L.; Wildung, M. R.; Croteau, R.; Scott, A. I. Protein Expr. Pur 1998, 13, 90-96.
8. Peters, R. J.; Flory, J. E.; Jetter, R.; Ravn, M. M.; Lee, H.-J.; Coates, R. M.; Croteau, R. B. Biochemistry 2000, 39, 15592 -15602.
9. Cyr, A.; Wilderman, P. R.; Determan, M.; Peters, R. J. J. Am. Chem. Soc. 2007, 129, 6684-6685.
10. Prisic, S.; Peters, R. J. Plant Physiol. 2007, 144, 445-454.
11. Smith, M. W.; Yamaguchi, S.; Ait-Ali, T.; Kamiya, Y. Plant Physiol. 1998, 118, 1411-1419.
12. Richter, S.; Lamppa, G. K. Proc. Natl. Acad. Sci. U. S. A. 1998, 95, 7463-7468.
13. Hart, E. A.; Rice University: Houston, Tex., 2001, p 144.
14. Matsuda, S. P. T.; Hart, E. A. U.S. Pat. No. 7,238,514 2004.
15. Stofer Vogel, B.; Wildung, M. R.; Vogel, G.; Croteau, R. J. Biol. Chem. 1996, 271, 23262-23268.
16. Sun, T. P.; Kamiya, Y. Plant Cell 1994, 6, 1509-1518.
17. Yamaguchi, S.; Sun, T. P. K., H.; Kamiya, Y. Plant Physiol. 1998, 116, 1271-1278.

What is claimed is:

1. A method comprising:
providing one or more plastid proteins comprising a signal sequence and one or more chloroplast processing enzymes; and
allowing at least one of the one or more chloroplast processing enzymes to cleave at least a portion of the signal sequence from at least one of the one or more plastid proteins.

2. The method of claim 1 wherein at least one of the one or more plastid proteins is an untruncated protein.

3. The method of claim 1 wherein at least one of the one or more plastid proteins is a diterpene synthase.

4. The method of claim 1 wherein the step of allowing at least one of the one or more chloroplast processing enzymes to cleave at least a portion of the signal sequence from at least one of the one or more plastid proteins increases the solubility in water of the at least one of the one or more plastid proteins.

5. The method of claim 1 wherein the system comprises a geranylgeranyl diphosphate synthase, a hydroxymethylglutaryl CoA reductase, a diterpene synthase, and a chloroplast processing enzyme.

6. The method of claim 1 wherein the chloroplast processing enzyme is derived from a source selected from the group consisting of an Arabidopsis thaliana and Abies grandis.

7. The method of claim 1 wherein the chloroplast processing enzyme and the one or more plastid proteins are derived from the same organism.

8. The method of claim 1 further comprising allowing the at least one of the one or more plastid proteins to catalyze a reaction.

9. The method of claim 1 further comprising allowing an increased yield of product to be derived from the plastid proteins.

10. The method of claim 8 wherein the reaction synthesizes one or more diterpene hydrocarbons.

11. The method of claim 10 wherein at least one of the one or more diterpene hydrocarbons is paclitaxel.

12. The method of claim 1 further comprising allowing one or more competing enzymes to be suppressed.

13. A system comprising:
one or more plastid proteins; and
one or more chloroplast processing enzymes.

14. The system of claim 13 wherein at least one of the one or more plastid proteins comprises a signal sequence.

15. The system of claim 13 wherein at least one of the one or more plastid proteins is an untruncated protein.

16. The system of claim 13 wherein at least one of the one or more plastid proteins is a diterpene synthase.

17. The system of claim 13 wherein the system comprises a geranylgeranyl diphosphate synthase, a hydroxymethylglutaryl CoA reductase, a diterpene synthase, and a chloroplast processing enzyme.

18. The system of claim 13 wherein the chloroplast processing enzyme is derived from a source selected from the group consisting of an Arabidopsis thaliana and Abies grandis

19. The system of claim 13 wherein the chloroplast processing enzyme and the one or more plastid proteins are derived from the same organism.

20. The system of claim 13 wherein the system further comprises P₄₅₀-dependent oxidases.
